(19) United States
(12) Patent Application Publication
(10) Pub. No.: US 2009/0104122 A1
(43) Pub. Date: Apr. 23, 2009

(54) DETECTION OF MEMBRANE ANDROGEN RECEPTOR (mAR) AGENTS

(75) Inventors: Nikolas Bacopoulos, Stonington, CT (US); Konstantinos Alevizopoulos, Neo Irakleio (GR); Robert L. Buchanan, Boston, MA (US); Christos Stournaras, Ag Myronas (GR)

Correspondence Address: EDWARDS ANGELL PALMER & DODGE LLP, P.O. BOX 55874, BOSTON, MA, 02205, US

(73) Assignee: MEDEXIS S.A., Athens (GR)

(21) Appl. No.: 12/294,209

(22) PCT Filed: Mar. 30, 2007
(86) PCT No.: PCT/US07/07913

§ 371 Date: Dec. 18, 2008

Related U.S. Application Data
(60) Provisional application No. 60/788,878, filed on Apr. 3, 2006.

(30) Foreign Application Priority Data
Apr. 3, 2006 (GR) 2006/0100207

(51) Int. Cl. A61B 10/00; G01N 33/566; G01N 33/542; C12Q 1/68

(52) U.S. Cl. 424/9.2; 436/501; 435/7.23; 435/7.21; 435/6; 435/7.9

(57) ABSTRACT

Disclosed are methods to detect agents that modulate a membrane androgen receptor (mAR). In one embodiment, the invention provides a method to detect and optionally identify mAR binding ligands. One assay according to the invention detects agents that selectively bind the mAR relative to binding to the classical intracellular androgen receptor (iAR or AR). The invention has a wide spectrum of useful applications including providing a method of screening libraries for chemical entities that preferentially bind the mAR.

### RELATED APPLICATIONS

This application claims the benefit of U.S. provisional patent application No. 60788,878, filed Apr. 3, 2006, and Greek patent application No. GR2006/0100207, filed Apr. 3, 2006. The entire contents of the aforementioned patent applications are incorporated herein by reference.

### FIELD OF THE INVENTION

The invention generally relates to the detection of ligands and other agents that modulate a membrane androgen receptor (mAR). More particularly, the invention provides a method to detect such agents. A preferred assay of the invention detects ligands that selectively bind the mAR relative to binding to an intracellular androgen receptor (iAR or AR). The invention has a wide spectrum of useful applications including providing a method for screening chemical libraries for entities that specifically bind the mAR.

### BACKGROUND

There is recognition that androgen activity is mediated through binding to an intracellular androgen receptor (referred to herein as iAR or AR). The iAR is said to be a member of the nuclear receptor superfamily that functions as a ligand-activated transcription factor. Androgen effects on cells that express the iAR have been reported to include binding to the receptor. Additional effects include androgen internalization, dimerization, nuclear translocation, nucleic acid binding, and activation/repression of androgen sensitive genes. Androgen mediated gene expression, acting through the AR, typically takes several hours. See generally, Pietras, R J. et al. (2001) Endocrine 14: 417-427, Kumar, M V (1998) Prog. Nucleic Acids Res. Mol. Biol. 59: 289-306; and references cited therein.

30. Certain androgen derivatives such as dihydrotestosterone (DHT) are thought to be natural AR ligands. Attempts to develop agents that mimic or inhibit these effects have been reported. For example, many small molecule AR modulators include cyproterone acetate, flutamide, chloramidone, bicalutamide, as well as certain quinoline and quinolinone derivatives. See e.g., PCT/IB2005/002592 (WO 2006/024931); PCT/US2004/038859 (WO 2005/052118); PCT/2004/027483 (WO 2005/018573); PCT/2005/013775 (WO 2005/105091); PCT/US2005/029592 (WO 2006/026196); and PCT/IB2005/002592 (WO 2006/024931); and references disclosed therein.

There is increasing recognition that the biological activity of at least some androgens is much more rapid (within minutes) than can be accounted for by AR binding (hours). Along these lines, there have been reports of “non-classical” steroid binding entities ie., binding entities that do not include the AR. See Papakonstanti, E. et al. (2003) Molecular Endocrinology 17: 870; Koukouitaki, S. B. et al. (1999) Mol. Med. 5: 731; and references cited therein.

More specifically, there are reports of androgen membrane binding sites in cells that express minimal or no iAR. Such cells include, but are not limited to, T lymphocytes, monocytes, ostoblasts, prostate tissues and certain cell lines. Reported common elements involved in the modulation of the mAR include increases in intracellular calcium, an increase in detectable prostate specific antigen (PSA), an increase in cell death (apoptosis) and substantial actin reorganization. See Gorczynska, E. and Handelsman, D J (1995) Endorcrinology 136: 2052; and Kampa, M. et al. (2002) FASEB J. 16: 1429.

The distribution and function of the mAR has been reviewed. See Kampa, M. E. Castanas (2006) in Mol. And Cell. Endocrinology 246: 76; and references disclosed therein. In brief, mAR activation is thought to play a key role in actin reorganization, apoptosis and modulation of intracellular calcium levels. It has been proposed that the mAR could represent a new therapeutic target. See Kampa, M. E. and Castanas (2006), ibid; Hatzoglou, A. et al. (2005) J. of Clinical Endocrinology & Metabolism, 90: 893; Kampa, M. et al. (2002), ibid; Stathopouos, E. et al. (2003) BMC Clinical Pathology 3:1; and references cited therein.

It would be desirable to detect agents that modulate the mAR. It would be more desirable to have a method of detecting agents that preferentially modulate the mAR, relative to any modulation of the AR. More preferred methods would be able to detect receptor binding ligands that specifically bind the mAR relative to binding to the AR.

### SUMMARY OF THE INVENTION

The invention generally relates to methods that can detect agents that modulate (increase or decrease) a membrane androgen receptor (mAR). More particularly, the invention relates to methods that can be used to detect agents that preferentially modulate the mAR, relative to modulation of the intracellular membrane receptor (iAR or AR). More particular methods can be employed to detect and identify ligands from that specifically bind the mAR relative to the AR. The invention has a wide spectrum of useful applications including providing methods that can be used to screen chemical libraries for molecules that specifically bind the mAR.

Illustrative agents are receptor binding ligands and like molecules. Such agents can be identified by using one or a combination of the in vitro and in vivo methods as described herein. Choice of a particular method (or combination of methods) according to the invention will be guided by recognized parameters such as the number of agents to be analyzed, the specificity and selectivity required, and the number of methods that can be performed by a user.

Accordingly, and in one aspect, the invention provides a method of detecting an agent that can modulate (increase or decrease) function of mAR. In one embodiment, the method comprises at least one of, and preferably all of, the following steps:

a. contacting the mAR with a detectably-labeled androgen compound under conditions that permit binding of the mAR and the compound to form a first complex,
b. contacting the first complex with the agent under conditions that permit binding between the agent and the mAR to form a second complex; and
c. detecting any release of the detectably-labeled androgen compound from the first complex as being indicative of the presence of the agent.

It will be appreciated that the order in which a detectably-labeled androgen compound and agent is employed in the invention is not important so long as intended results are achieved. Thus in one embodiment, the method can be adapted so that the first complex is formed, sometimes with excess androgen, before adding the agent to be tested (saturation format). Alternatively, the agent and the detectably-labeled androgen can be added at the same time to form the first complex (competitive format). In yet another method embodiment, the detectably-labeled androgen is added after addition of the agent to screen.

Thus in one embodiment, the invention encompasses detecting an agent that modulates function of the mAR that includes at least one of and preferably all of the following steps:

a. contacting the mAR with a detectably-labeled androgen compound and the agent (by mixing them together or by combining them one at a time, for instance) under conditions that permit binding of the mAR and the compound to form the first complex; and
b. detecting a decrease in the level of detectably-labeled androgen compound bound to the first complex as being indicative of the presence of the agent.

In the foregoing invention embodiment, the first complex is formed in the presence of the agent to be tested. The detectably-labeled androgen and agent compete for binding to the mAR. Any complex formed between the mAR and the agent is referred to herein as a “second complex”. In this embodiment, a decrease in the level of detectably-ladled androgen compound (e.g., more second complex detected than first complex) is taken to be indicative of agent that modulates mAR. Although the order of addition of screening components is not always important, it will often be useful to form the first complex prior to addition of the agent to be tested.

Although it will not always be required for optimal practice the invention (for example, where the receptor activity of a particular agent or agents known), it will often be helpful to include one or a combination of suitable controls. Generally, a “suitable control” will give information about the activity of an agent or agents compared to the absence of such agent or agents. Alternatively, or in addition, a “suitable control” will give information about the activity of an agent or agents with respect to the AR. Prudent use of such controls can provide many practical benefits including increasing the likelihood of detecting agent that can specifically bind the mAR. As discussed, particular agents of interest will demonstrably increase mAR activity and preferably also show negligible or no activity in one or combination of controls assays as provided herein. By way of example, a particular agent of interest can effectuate, either directly or indirectly, a substantial increase in the release of the detectably-labeled androgen compound from the first complex. Preferably, that agent will show negligible or no activity in a suitable control assay designed to detect and optionally quantitate any capacity of that agent to modulate the AR.

Accordingly, it will often be useful to perform one or more suitable control assays such as those designed to detect modulation of the AR. In a particular embodiment, the capacity of agent to increase mAR activity is evaluated and compared to the capacity of that agent to increase or decrease AR function. More specific control assays involve contacting the agent with a suitable cell or subcellular fraction (e.g., membrane, nuclear or cytosolic fraction) of cells known to express the AR. Such a control assay involves detecting activity of the agent. That activity can be monitored in the control assay directly (e.g. by monitoring receptor binding) or it can be monitored indirectly (eg., by monitoring a molecule “downstream” from the AR; by monitoring gene transcription, translation, or AR specific protein expression). An illustrative control along these lines is one that is designed to detect and optionally quantify capacity of the agent to bind the AR as a receptor ligand. Preferred agents according to the invention will exhibit negligible or no capacity to increase AR activity by one or a combination of the control assays disclosed herein. A highly preferred agent shows substantial activity in one or more of the mAR assays of the invention and shows negligible or no activity in any of the AR control assays.

In one embodiment of a suitable control assay of the invention, at least one and preferably all of the following steps are conducted:

i. contacting the iAR with the detectably-labeled androgen compound under conditions that permit binding of the iAR and the compound to form a third complex; and
ii. contacting the third complex with the agent to be tested under conditions that permit binding between the compound and the third complex. A preferred agent will effectuate negligible or no release of the detectably-labeled androgen from the third complex.

In another suitable control embodiment, the iAR is contacted with the detectably-labeled androgen compound and the agent. Preferred is contact under conditions that permit binding of the iAR and the agent to form the third complex. In this illustration of the invention, the control is designed to effectuate formation of the third complex in a competitive format. Any complex formed between the iAR and the agent is referred to herein as a “fourth complex”. Alternatively, the agent to be tested can be contacted with the iAR under conditions that favor formation of the third (or fourth) complex, prior to contact between the iAR and the detectably-labeled androgen.

The invention is flexible and can be modified as needed to suit an intended use. Thus for instance, the detectably-labeled androgen compound used in one or the aforementioned controls can be the same or different from the one used in a selected mAR assay. Choice of a detectably-labeled androgen to use in a given control will be guided by recognized parameters such as the specific control assay selected, the number of agents to be tested, and the assay sensitivity required.

Although not always necessary for optimal use of the invention, it will sometimes be helpful to conduct a method to test the capacity of an agent to modulate mAR function essentially in parallel with a control (or controls) to detect any capacity of that agent to modulate the AR. Thus in one embodiment, one or a combination of the mAR screening assays are conducted essentially in parallel with one or a combination of control assays for detecting capacity of agent to modulate the AR. As will be appreciated, it may not always be necessary to conduct a particular mAR assay and a control assay in parallel (ie., at the same time). For instance, in embodiments in which the activity of the AR with respect to a given agent(s) is relatively well known it may not be necessary to practice the control each time a selected mAR assay is performed. Alternatively, and in embodiments in which large numbers of agents are to be screened (eg., where large chemical libraries are to be examined), then practice of one or a combination of control assays may not be needed to achieve optimal results. For instance, it may be useful to screen the agents first by detecting potential to activate the mAR and then to follow-up subsequently with further testing using one or a combination of the control assays.

For many invention embodiments, it will be useful to include at least some control assays for monitoring AR modulation, especially in embodiments in which the invention is used according to a high- or ultra-high throughput screening (HTS) format. Such a format is particularly useful for analyzing large numbers of candidate agents for mAR activation potential.

In embodiments in which a control assay is used to detect the third complex (formed between the iAR and the detectably-labeled androgen compound), the method will further include the step of detecting release of the detectably-labeled androgen compound from the third complex in the presence of the agent. Preferably, such an agent will exhibit negligible or no release of the labeled androgen compound from the third complex.

These and other uses and advantages of the invention will be apparent from the discussion and Examples below.

### BRIEF DESCRIPTION OF THE DRAWINGS

FIGS. 1A-1B are graphs showing saturation and displacement binding of tritiated-testosterone on membranes of cells that express the mAR (LNaCP) cells.

FIG. 2A-B are graphs showing modification and polymerized actin in DU-145 cells after testosterone treatment.

FIG. 3A-C are photomicrographs showing morphological analyses of androgen effects on actin cytoskeleton organization.

FIG. 4 is a graph showing cell death (apoptosis) in cells exposed to various compounds.

FIGS. 5A-B are graphs showing activity of various signal molecules following exposure to a testosterone BSA conjugate.

FIG. 6A is a representation of a gel and FIG. 6B is a graph. Both show RhoA and ROCK signaling molecules are activated when DU-145 cells are treated with testosterone-BSA conjugate.

### DETAILED DESCRIPTION OF THE INVENTION

As discussed, the present invention can be used to detect agents that increase or decrease mAR binding. Preferred agents are receptor ligands that increase activation of the mAR, either preferentially or exclusively, relative to potential to modulate the AR. Preferred receptor ligands specifically bind the mAR with negligible or no capacity to bind and activate the AR.

As used herein, an “agent” including plural forms refers to a moiety capable of associating with the mAR or AR (or both), either by non-covalent or covalent interaction. Preferred agents preferentially associate, either directly or indirectly, with the mAR as determined by one or a combination of the methods disclosed herein. A more preferred agent is a “ligand”. A “ligand” refers to a particular agent that binds the mAR (or AR), typically through non-covalent interactions. Preferred ligands have a receptor binding constant sufficient to allow detection of binding by one or more of the assays disclosed herein. Illustrative of such assays include those that directly or indirectly monitor formation or breakdown of a receptor binding complex, detection of second messenger, detection of apoptosis, detection of prostate specific antigen (PSA), detection of intracellular calcium, a binding assay to measure protein-ligand binding, an immunoassay to measure antibody-antigen interactions or rearrangement of the cytoskeleton. An illustrative ligand according to the invention is a particular androgen such as testosterone and derivatives thereof (e.g., DHT). Other suitable ligands include antibody, antigen, enzyme, peptide, polypeptide, peptoid, nucleic acid, steroid, or small molecule ligands that bind the mAR. Preferred ligands specifically bind the mAR, typically to form a receptor-ligand complex, with negligible or no potential to interact with the AR.

As used herein, the term “second messenger” refers to a molecule, generated or caused to vary in concentration by the activation of the mAR or AR. The term “change in the level of a second messenger”, refers to an increase or decrease of at least about 10% in the detected level of a given second messenger relative to the amount detected in an assay performed in the absence of a candidate modulator. Illustrative second messenger (signaling) molecules are provided herein.

By the term, “specific binding” or a similar term is meant a molecule (e.g, an agent) disclosed herein which binds another molecule (e.g., a receptor), thereby forming a specific binding pair. Preferably, the molecule does not recognize or bind to other molecules as determined by, e.g., Western blotting ELISA, RIA, mobility shift assay, enzyme-immuno assay, competitive assays, saturation assays or other protein binding assays know in the art. See generally, Ausubel, et al supra; Harlow and Lane in, Antibodies: A Laboratory Manual (1988) and references cited therein for examples of suitable immunological methods and methods for detecting specific binding between molecules. In preferred embodiments, “specific binding” between and agent and the mAR will be manifested by K_{d} of 1 mM or less, generally in the range of 0.01 nM to about 100 nM, preferably between from about 0.1 nM to about 10 nM. Various standard methods for determining K_{d} are disclosed herein.

In embodiments in which an mAR assay of the invention detects one or more of a second messenger, intracellular calcium, PSA secreation, or rearrangement of the cytoskeleton, the detection will take place during the time in which the mAR is believed to exert maximal effects, ie., less than about 3 hours, preferably less than about 1 to about 2 hours, more preferably between from about a few minutes to about 30 minutes. In contrast, apoptotic effects can be detected after a much longer time ie., between from about several hours up to a few days, for instance, one, two, three or four days up to about a week.

An agent, particularly an mAR receptor ligand that associates (eg., binds non-covalently) with the mAR and mimics the effects of an natural mAR ligand (e.g., testosterone) is referred to herein as an “mAR agonist”. In contrast, an agent that inhibits effects of an natural mAR ligand (e.g., testosterone) is called an “mAR antagonist”.

More broadly, an mAR agonist of the invention will modulate (in this case increase) one or more biological responses mediated by the mAR by at least about 2-fold, preferably about 5-fold, more preferably about 10-fold and most preferably about 100-fold or more (i.e., about 150-fold, 200-fold, 250-fold, 500-fold, 1000-fold, up to about 10,000-fold), as compared to a suitable control such as binding of a natural ligand (eg., testosterone or DHT). On the other hand, an mAR antagonist according to the invention will modulate (in this case decrease) one or more of the biological responses mediated by the mAR at least about 2-fold, preferably about 5-fold, more preferably about 10-fold and most preferably, about 100-fold or more (i.e., 150-fold, 200-fold, 250-fold, 500-fold, 1000-fold, up to about 10,000-fold), as compared to the response in the presence of the natural ligand.

The word “androgen” including plural forms is used herein to encompass any natural, synthetic, semi-synthetic or recombinant material that stimulates the development of masculine characteristics in vertebrates. Preferred androgens typically demonstrate capacity to modulate the mAR by one or a combination of known assays such as those discussed below. More particular androgens include those natural or synthetic compounds that control activity of the accessory male sex organs. Androgens are sometimes known as “androgenic hormones” or, in some cases, “testoids”.

A more particular androgen according to the invention exhibits significant activity in an accepted in vitro or in vivo assay. See, for instance, assays for testing AR modulation in PCT/IB2005/002592 (WO 2006/024931); PCT/US2004/038859 (WO 2005/052118); PCT/2004/027483 (WO 2005/018573); PCT/2005/013775 (WO 2005/105091); PCT/US2005/029592 (WO 2006/026196); and PCT/IB2005/002592 (WO 2006/024931); and references disclosed therein.

A suitable in vitro assay for identifying an androgen is what is referred to in the field as a “radioligand displacement assay”. Briefly, the assay detects and preferably measures capacity to displace an AR receptor ligand (eg., DHT). Various manipulations of this general test can be performed to identify IC₅₀, K_{d}, and Kᵢ values for a given agent. See one or more of PCT/IB2005/002592 (WO 2006/024931); PCT/US2004/038859 (WO 2005/052118); PCT/2004/027483 (WO 2005/018573); PCT/2005/013775 (WO 2005/105091); PCT/US2005/029592 (WO 2006/026196); and PCT/IB2005/002592 (WO 2006/024931); and references disclosed therein, for example, PCT/US2005/029592 (WO 2006/026196), for instance, at pgs. 54-56. A suitable in vivo assays measure the prostate gland (and, if needed, accessory organs) following testicle ablation in a suitable animal model. See also Chang et al. (1984) J. Steroid Biochem. 20:1-17; and Chang et al. (1992) PNAS 89: 5546 for other preferred assay formats. Thus an “androgen” according to the invention will exhibit good activity in one or more of these assays.

A “derivative” of an androgen is one that bears a structural and functional relationship to testosterone but which also bears one or more new chemical groups. Such groups can include but are not limited to saturation groups, desaturation groups, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), other steroids (including estrogens, other androgens, cortisone, ecodysone, etc.), alkaloids (including ergots, vinca, curare, pyrollizdine, and mitomycines), organometallic compounds, hetero-atom bearing compounds, amino acids, and nucleosides.

More particular androgens and androgen derivatives include, without limitation, the adrenal androgens ie., any of the 19-carbon steroids synthesized by the adrenal cortex. Other androgens include testosterone, dihydrotestosterone (DHT), particularly 5-α-dihydrotestosterone, dehydroepiandrosterone (DHEA, also called dhydroisoandrosterone or dhydroandrosterone), androstenedione, androstanediol; and natural, synthetic or semi-synthetic derivatives and salts thereof. An “androgen” of the invention can be provided as a salt, preferably one that is pharmaceutically acceptable. Examples of such salts include an acid addition salt such as an inorganic acid addition salt, e.g., a hydrochloride, sulfate, or phosphate salt, or as an organic acid addition salt such as an acetate, maleate, fumarate, tartrate, or citrate salt. Pharmaceutically acceptable salts can also include metal salts, particularly alkali metal salts such as a sodium salt or potassium salt; alkaline earth metal salts such as a magnesium or calcium salt; ammonium salts such an ammonium or tetramethyl ammonium salt; or an amino acid addition salts such as a lysine, glycine, or phenylalanine salt. Additional salts include sodium, chloride, bromide, fumarate, citrate, proprionate, benzoate, cypionate, and caproate salts.

Many derivatives of testosterone are known and include D(-)-Norgestrel, epitestosterone, testosterone 17-benzoate, testosterone 17-phenylpropionate, testosterone 17β-cypionate, testosterone 3-(O-carboxymethyl)oxime, an oxime of testosterone such as testosterone 3-(O-carboxymethyl)oxime: BSA, testosterone 3-(O-carboxymethyl)oxime: BSA-fluorescein isothiocyanate conjugate, testosterone acetate, testosterone β-D-glucuronide, testosterone β-D-glucuronide sodium salt, testosterone enanthate, testosterone isocaproate, testosterone propionate, testosterone-d3, 17-α-2-carboxy-ethyl-testosterone-γ-carboxy-ethyl-lactone, testosterone acetate, testosterone-beta-D-glucuronide, and 17-α-(2-carboxyethyl) testosterone-γ-lactone, 5α-Androstan-17β-ol-3-one, androstanolone 17-benzoate, dihydrotestosterone propionate, methyltrienolone, androstanolone, dihydrotestosterone, 17-Hydroxyandrostan-3-one, hermalone, androstanolone acetate, 5β-dihydrotestosterone, androstanolone propionate, dihydrotestosterone enanthate, mestanolone, 17-bromoacetoxydihydrotestosterone, and emdisterone.

See also Hauptman, H. (2003) Steroids 68: 629 (describing 1α- and 17α-aminoalkyl derivatives of dihydrotestosterone). See also the National Library of Medicine (8600 Rockville Pike, Bethesda, Md. 20894 (USA)) PubChem Database disclosing additional derivatives of testosterone.

Other particular testosterone derivatives within the scope of the present invention include those molecules in which, for instance, testosterone or DHT is joined to a much larger protein molecule, either directly or indirectly through a suitable linker. An illustrative protein for this purpose will have a molecular weight of from between about 30 kD to about 100 kD, preferably between from about 40 kD to about 80 kD, and more preferably around 60 kD. An illustrative protein is a serum albumin such as bovine serum albumin (BSA) or human serum albumin (HSA). Illustrative albumins, including suitable fragments and derivatives thereof, are discussed below. Molecular weights are determined by standard means including gel electrophoresis. If desired, the protein joined to the testosterone may itself be detectable, for instance, a GFP.

Still further androgens in accord with the present invention are disclosed at the internet website of the U.S. Food and Drug Administration (USFDA). In particular, the Endocrine Knowledge Disrupter Base (EKDB) features some 2000 naturally-occurring and synthetic compounds that are reported to have androgenic (and/or estrogenic activity). Data are included for biological assays that measure such activities. See also Fang, H. et al. (2003) Chem. Res. Toxicol. 16: 1338 for a review of androgens and in silico methods for detecting same.

A preferred androgen for many of the suitable control assays disclosed herein is testosterone or DHT. However, it will be appreciated that other androgens, particularly testosterone derivatives can be used (eg., methyltrienolone) in one or more of such assays. In general, DHT will be selected on grounds that it is a natural AR ligand.

As discussed, it is an object of the present invention to detect and optionally identify agents, for instance, receptor binding ligands, that can bind the mAR. Typical of such agents are mAR agonists. By “membrane androgen receptor” including plural forms and abbreviated forms such as “mAR” is meant an androgen sensitive receptor that exerts an androgen mediated effect “rapidly” ie., within three hours, preferably less than about 1 to about 2 hours, more preferably in less than an hour, still more preferably between from about a few minutes (one or two, for instance) up to about 30 minutes. See Kampa, M. E. Castanas (2006), ibid. (reviewing a variety of acceptable mAR assays)

For instance, in one mAR agent detection format, one or more of actin reorganization, intracellular calcium release, or PSA release can be measured to identify agents that bind the mAR between from about a few minutes to about 3 hours. Binding to the mAR is believed to be associated with “rapid” effects, including calcium mobilization, secretion, and actin reorganization. Other methods for detecting and analyzing the mAR have been reported in certain cells, cell lines and tissues. See eg., Porto, C. S. et al. (1995) in Steroid Biochem. Mol. Biol. 53: 561.

By “tissue” is meant an aggregate of cells that perform a particular function in an organism. The term “tissue” as used herein refers to cellular material from a particular physiological region. The cells in a particular tissue can comprise several different cell types. A non-limiting example of this would be prostate gland and surrounding tissue that further comprises, for instance, neurons, as well as capillary endothelial cells and blood cells, all contained in a given tissue section or sample. In addition to solid tissues, the term “tissue” is also intended to encompass non-solid tissues, such as blood and lymph.

A preferred approach for identifying cells, cell lines and tissues that express the mAR include contacting same with a particular derivative of testosterone in which the steroid ring is linked to a macromolecule. Such a molecular design is intended to greatly increases the molecular weight of the androgen. In doing so, the steroid is prevented from being internalized by the classical intracellular androgen membrane receptors (iAR). In a more specific and suitable approach, a detectably-labeled testosterone-serum albumin conjugate is used as a probe to identify the mAR. See eg., Kampa, M. E. and Castanas (2006), ibid; Hatzoglou, A. et al. (2005), ibid; Kampa, M. et al. (2002), ibid; Stathopouos, E. et al. (2003), ibid (for disclosure relating to making and using such mAR probes).

See also Dambaki, C. et al. (2005) BMC Cancer, 5: 148; as well as PCT/IB03/02785, U.S. Patent Publication No. 20050245442 and Greek Patent Application No. 20020100335 to E. Castanas (University of Crete Medical School (Heraklion, Greece)). A preferred testosterone serum albumin conjugate for the detection and characterization of the mAR is Test-BSA.

By using one or a combination of suitable methods, it has been possible to detect the mAR in a variety of cells and tissues e.g., prostate carcinoma tissues, prostate cancer LNCaP cells, DU-145 prostate cancer cells, and the human breast cancer cell line T47D. See Kampa, et al. (2005), ibid; Kampa et al. (2002); ibid; and Hatzoglou et al. (2005), ibid. In contrast, it is believed that certain tissue samples such as non-tumoral and hyperplastic cells express low or nearly undetectable levels of the mAR. Stathopoulos et al. (2003), ibid. It has been reported that LNCaP cells express mAR and iAR receptor molecules while the DU-145 cell line expresses only the mAR (ie., the iAR was not detectable).

Using these and related approaches for detecting the mAR, it is possible to screen nearly any desired cell, cell line or tissue sample for presence of the mAR. Samples in which the mAR is detected can be used in accord with the methods described herein. For example, and in one embodiment, the LNCaP, T47D, or DU-145 cell lines are used as a source of mAR. In embodiments in which it is useful to have cells in which mAR (but not AR) is expressed, use of the DU-145 cell line will be especially preferred. Suitable cells, cell lines and tissue samples can be obtained from a variety of sources including the German Collection of Microorganisms and Cell Cultures (Brauschweig, Germany) and the American Type Culture Collection (ATCC) (Manassas, Va.).

Examples of cells which can be screened for presence of the mAR (or AR) include suitable eukaryotic cell types such as tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Other suitable cells include Jurkat T cells, NIH3T3 cells, CHO, COS, etc. See the ATCC cell line catalogue list.

As discussed, it is an object of the invention to provide a method that can detect and optionally identify a receptor ligand that can activate the mAR. Preferred practice involves contacting the mAR with a detectably-labeled androgen compound (e.g., ³H-DHT or other suitable testosterone derivative that binds AR) under conditions that permit binding of the mAR and the compound to form a first complex. Subsequently, the first complex is contacted with the agent under conditions that permit binding between the agent and the mAR to form a second complex. Preferably, any release of the detectably-labeled androgen compound from the first complex is detected and is taken to be indicative of the presence of the agent.

By the phrase “conditions that permit binding of the mAR and the agent” including the singular forms is meant conditions that generally favor binding of testosterone, DHT or a testosterone derivate such as testosterone 3-(O-carboxymethyl)oxime: BSA to the mAR. Such conditions will encompass temperature, salt concentration, pH and protein concentration under which, for instance, testosterone 3-(O-carboxymethyl)oxime: BSA binds the mAR. Exact binding conditions will vary depending upon the nature of the assay, for example, whether the assay uses viable cells or only membrane fraction of cells. However, because mAR is a cell surface protein, favored conditions will generally include physiological salt (about 90 mM) and pH (about 7.0 to 8.0). Temperatures for binding can vary from 15 C. to 37 C., but will preferably be between room temperature and about 30 C. The concentration of the agent to be detected will also vary, but will preferably be about 0.01 nM to 100 micromolar.

As used herein, the term “membrane fraction” refers to a preparation of cellular lipid membranes comprising an mAR or AR. As the term is used herein, a “membrane fraction” is distinct from a cellular homogenate, in that at least a portion (i.e., at least 10%, and preferably more) of non-membrane-associated cellular constituents has been removed. The term “membrane associated” refers to those cellular constituents that are either integrated into a lipid membrane or are physically associated with a component that is integrated into a lipid membrane.

More specific conditions for binding ligand to the mAR receptor have been reported. See Hatzoglou et al. (2005) J. Clin. Endocrinol Metab. 90: 893; and Kampa, M. et al. (2002), supra; in which each reference describes specific conditions for binding DHT, testosterone and testosterone 3-(O-carboxymethyl)oxime: BSA conjugate to the mAR.

Thus by the phrase “conditions that permit binding of the AR and agent” including similar phrases is meant those conditions which generally permit binding between the mAR and testosterone, DHT or as testosterone 3-(O-carboxymethyl)oxime: BSA.

By the phrase “detectably-labeled androgen” is meant an androgen that includes at least one of the following: a radioisotope, fluorophore, a quencher of fluorescence, an enzyme, an affinity tag, or a chemiluminescent, phosphorescent or chromophoric moiety. The detectable label may present on the steroidal ring of the androgen (eg., at one or more of the 1, 3, 7, 11 and 15 carbon positions) or it may appear on another group, for instance, on a linker or an amino acid sequence bound to the androgen or the linker. An example of such a linker is carboxy-methyl ether moiety attached to the steroid ring through use of carboxydiimide. More particular linkers include an oxime group. By way of illustration, a detectably-labeled androgen includes a testosterone oxime and a testosterone serum albumin conjugate, in which one or more detectable labels is attached to a suitable oxime linker or to the amino acid sequence bound to the linker. See, for example, PCT/IB03/02785, U.S. Patent Publication No. 20050245442 and Greek Patent Application No. 20020100335 for more information regarding making and using these particular androgens.

i. Assay Detection Formats

A wide variety of detectable labels for use with the invention have been reported. See generally W. T. Mason in Fluorescent and Luminescent Probes for Biological Activity: A Practical Guide to Technology For Quantitative Real-Time Analysis, 2^{nd} Ed. (Academic Press, 1999). Exemplary labels for labelling amino acid sequences include fluorescein, rhodamine red, FITC and Oregon green. A variety of other suitable fluorescent, luminescent and chromogenic probes can be obtained from InVitrogen (1600 Faraday Avenue, Carlsbad, Calif. 92008 (USA)).

Use of additional fluorescent labels are within the scope of the present invention.

For instance, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, and LC Red 705. Suitable optical dyes are described in the 1996 Molecular Probes Handbook by Richard P. Haugland, hereby expressly incorporated by reference. Suitable fluorescent labels also include, but are not limited to, green fluorescent protein (GFP; Chalfie, et al., Science 263(5148):802-805 (Feb. 11, 1994); and EGFP; Clontech-Genbank Accession Number U55762), blue fluorescent protein (BFP; 1. Quantum Biotechnologies, Inc. 1801 de Maisonneuve Blvd. West, 8th Floor, Montreal (Quebec) Canada H³H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), .beta.-galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558).

Thus in one invention embodiment, an androgen such as testosterone is linked to a fluorescent entity such as the GFP protein or a detectable fragment thereof. In this example, the GFP is obtained from a renilla, ptilosarcus, or aequorea species of GFP.

Additionally suitable labels for some invention embodiments include: Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes) (Eugene, Oreg.), FITC, Rhodamine, and Texas Red (Pierce, Rockford, Ill.), Cy5, Cy5.5, Cy7 (Amersham Life Science, Pittsburgh, Pa.). Tandem conjugate protocols for Cy5PE, Cy5.5PE, Cy7PE, Cy5.5APC, Cy7APC can be found at drmr.com/abcon. Quantitation of fluorescent probe conjugation may be assessed to determine degree of labeling and protocols including dye spectral properties can be found at metazoa.com/UPL3419.

In one invention embodiment, the androgen is detectably-labeled with a secondary detectable label, either directly or indirectly (eg., via a linker). A secondary label is one that is indirectly detected; for example, a secondary label can bind or react with a primary label for detection, can act on an additional product to generate a primary label (e.g. enzymes), etc. Secondary labels include, but are not limited to, one of a binding partner pair; chemically modifiable moieties; nuclease inhibitors, enzymes such as horseradish peroxidase, alkaline phosphatases, lucifierases, etc.

By way of example, the secondary label is a binding partner pair. For example, the label may be a hapten or antigen, which will bind its binding partner. For example, suitable binding partner pairs include, but are not limited to: antigens (such as proteins (including peptides) and small molecules) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Preferred binding partner pairs include, but are not limited to, biotin (or imino-biotin) and streptavidin, and digeoxinin

In another invention embodiment, the secondary label is a chemically modifiable moiety. In this embodiment, labels comprising reactive functional groups are incorporated into the molecule to be labeled, usually an androgen. The functional groups can be added to the steroid ring directly or indirectly via a suitable linker or protein group such as serum albumin (including fragments thereof). The functional group can then be subsequently labeled (e.g. either before or after the assay) with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo- or hetero-bifunctional linkers as are well known (see 1994 Pierce Chemical Company catalog, technical section on cross-linkers, pages 155-200, incorporated herein by reference).

The methods and composition of the present invention may also make use of label enzymes. By label enzyme is meant an enzyme which may be reacted in the presence of a label enzyme substrate which produces a detectable product. Suitable label enzymes for use in the present invention include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

A variety of suitable methods for detecting label enzymes and other detectable labels have been described. See Harlow and Lane in, Antibodies: A Laboratory Manual (1988) and references cited therein (sandwich assays and the like). For instance, and in one embodiment, the detectable label is biotin or DNP in which the biotin moiety is detected using standard antibody detection methods. Alternatively, the detectable label is one or more of a radioactive isotope such as tritium, deuterium or carbon-14. An illustrative radioactively labeled androgen compound is (³H-DHT) or tritiated testosterone (³H-T). For instance, a specific tritiated testosterone compound is testosterone-[1,2,6,7-³H] (available from New England Nuclear, Beverly Mass. (USA)). Methods for tritiating androgens according to the invention are known and include, for instance, conventional isotope exchange reactions. Other suitable radioisotopes include ¹⁴C, ¹²⁵I, and ¹³¹I.

A detectable label may be detected indirectly, for instance by an antibody. That is, the antibody may be able to detect the androgen compound directly or indirectly through a tag bound to the compound. The tag is typically a binding partner of the antibody. Suitable binding pairs for use in embodiments using such antibodies include but are not limited to, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avid (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto.

Other suitable “tag” groups that can be used to detectably label an androgen compound of the invention include poly-histidine (poly-his) or poly-histidine-glycine (poly-his-gly) tags and nickel substrate; the Glutathione-S Transferase tag and its antibody substrate (available from Pierce Chemical); the flu HA tag polypeptide and its antibody 12CA5 substrate [Field et al., Mol. Cell. Biol., 8:2159-2165 (1988)]; the c-myc tag and the 8F9, 3C7, 6E10, G4, B7 and 9E10 antibody substrates thereto [Evan et al., Molecular and Cellular Biology, 5:3610-3616 (1985)]; and the Herpes Simplex virus glycoprotein D (gD) tag and its antibody substrate [Paborsky et al., Protein Engineering, 3(6):547-553 (1990)]. The production of tag-polypeptides by recombinant means when the tag is also a polypeptide is described below. Production of tag-labeled proteins is well known in the art and kits for such production are commercially available (for example, from Kodak and Sigma). Examples of tag labeled proteins include, but are not limited to, a Flag-polypeptide and His-polypeptide. Methods for the production and use of tag-labeled proteins are found, for example, in Winston et al., Genes and Devel. 13:270-283 (1999), incorporated herein in its entirety, as well as product handbooks provided with the above-mentioned kits.

Certain detectable androgen compounds of the invention may be biotinylated. Biotinylation of target molecules and substrates is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known (Id.).

Methods for labeling of proteins with radioisotopes are known in the art. For example, such methods are found in Ohta et al., Molec. Cell 3:535-541 (1999), which is hereby incorporated by reference in its entirety.

Production of proteins having tags by recombinant means is well known, and kits for producing such proteins are commercially available. For example, such a kit and its use is described in the QIAexpress Handbook from Qiagen by Joanne Crowe et al., hereby expressly incorporated by reference. Thus in one embodiment, a serum album is expressed recombinantly with one or more of the tags. Such a modified albumin can be joined to the androgen using standard methodologies, either directly or via a suitable linker.

By the phrase “release of the detectably-labeled androgen compound” is meant a detectable increase in the release of the androgen compound by virtue of the presence of agent in a particular assay, relative to a suitable control (e.g, release in the absence of the agent). Preferably, the release is at least about 20% higher relative to the control in which the agent is not added, preferably at least about 50% higher, more preferably at least about 80%, about 90% or about 95% or more. In this embodiment, the agent is a potential mAR agonist that can be isolated, identified and subjected to further testing according to the invention.

The following paragraphs A-E describe preferred methods for detecting and quantifying the capacity of an agent or agents (e.g., a library of agents) to modulate (increase or decrease) mAR function.

By “mAR function” is capacity of the receptor to modulate (increase or decrease) on or more of modulation of a signaling molecule as defined here, PSA secretion, modulation of calcium flux, and cytoskeleton reorganization. Preferably, the function is monitored within 3 hours after contact with a suitable compound agent (eg., DHT), preferably between from about a few minutes to about an hour, more preferably between from about a few minutes to 30 minutes.

A. Membrane Androgen Receptor (mAR) Binding

Release of detectably-labeled androgen can be quantified, for example, to confirm agonist activity of agent and/or to gauge the strength of that activity. Thus, in one embodiment the following specific mAR binding assays can be used to determine K_{d} of an agent.

First, suitable mAR-expressing cells (eg., DU-145 cells) are maintained in serum free medium for about 1-2 days. One or both of the following assays can be performed with the cells: (1) an mAR saturation analysis to determine K_{d} for tritiated DHT or (2) a competition binding assay to evaluate the ability of agent to compete with DHT for the mAR. Such assays are exemplified by the following discussion.

After maintaining in serum-free media, DU-145 cells can be contacted with media that includes a saturating amount of detectably-labeled DHT (eg., ³H-DHT) in amounts generally ranging from 0.05 nM to about 20 nM in the absence (total binding) or presence of a 100-fold excess of unlabeled DHT. For competitive assays, cell media containing about 0.1 nM to about 1 nM ³H-DHT and one of more agents to be tested (concentrations ranging from 10⁻¹⁰ M to 10⁻⁵ M) are added to the cells. Preferably, three replicates are used for each sample. After about 5 hours incubation at 37 C, preferably about 3-4 hours, an aliquot of the total binding media at each concentration of ³H-DHT is removed to estimate the amount of free ³H-DHT. Remaining media is removed, the cells washed to remove any unbound agent and cells lysed with Triton-X-100. Lysates are assayed for the amount of bound ³H-DHT using a standard scintillation counter. Any non-specific binding can be determined conventionally.

If desired, the assay can be performed with other suitable cells, such as LNCaP, or other suitable cells the express the mAR.

i. mAR Saturation Analysis

For the saturation analysis, the differences between the total binding and the non-specific binding, are normalized. Specific mAR receptor binding can be evaluated by standard Scatchard analysis to determine the Kd for ³H-DHT. As will be appreciated, a Scatchard analysis is a method of linearizing data from a saturation binding experiment to determine binding constants (K_{d}). One creates a “secondary” plot of specific binding/free radioligand concentration (Y axis) vs. specific binding (X axis). See eg., Robard, D. in Ligand Assay, J. Langon and J J. Clapp (eds.) (Masson Publishing U.S.A.), pp. 45-99 (1981).

Particular agents of interest will exhibit and K_{d} of less than about 1 micromolar, preferably between from about 0.01 nM to about 0.1 micromolar, more preferably between from about 0.1 nM to about 50 nM.

ii. mAR Competition Analysis

For receptor competition studies, data can be plotted as the amount of ³H-DHT (% of control in the absence of agent) remaining over the range of the dose-response curve for given agent. The concentration of any agent that inhibits 50% of the amount of the ³H-DHT bound in the absence of competing receptor ligand can be quantified (called IC₅₀) using standard methods. See, for example, Robard, D. (1981), supra; and PCT/US2004/027483. After correcting for any non-specific binding, IC₅₀ values can be determined. The IC₅₀ value is defined herein as the concentration of competing agent needed to reduce binding by about 50%.

Particular agents of interest will exhibit an IC₅₀ less than about 1 micromolar, preferably between from about 0.01 nM to about 0.1 micromolar, more preferably between from about 0.1 nM to about 20 nM.

B. Detection of Membrane Androgen Receptor (mAR) Signaling Molecules

As discussed, it is an object of the present invention to detect agents that modulate mAR receptor activity. Such activity can be detected at the level of receptor binding or after such binding (referred to as “downstream” activation of the mAR). Several downstream mAR signaling molecules have been reported including at least one of and preferably all of a focal Adhesion Kinase (FAK), phosphatidylinositol-3 (PI-3) kinase, cell division cycle 42 (cdc42) and ras-related C3 botulinum toxin substrate 1 (Rac 1) molecules. Activation of this particular pathway results in a rapid actin cytoskeleton rearrangement (controlling cell proliferation, secretion and motility, for instance). See Papakonstanti, E. et al. (2003) Mol. Endocrinology. 17:870.

More recently, actin cytoskeleton reorganization has emerged as one important outcome of mAR activation. See Papadopoulou, N. D. et al. (2004), 56th Meeting of HSBMB (Hellenic Society of Biochemistry and Molecular Biology) 25-27 Nov. 2004, Larissa, Greece.

Non-genomic androgen effects are initiated at the membrane level. This indicates, for instance, that specific secretory and signaling mechanisms (distinct from those downstream of the iAR). Androgen membrane binding sites (ie., the mAR) are present in LNCAP human prostate cells.

As will become apparent from the present disclosure, it is possible to activate the mAR of DU-145 cells by contacting the cells with testosterone-BSA. This contact induced significant actin remodelling followed by a decrease in cell migration, adhesion and invasion. As also shown, these effects were also induced through activation of a downstream signaling pathway involving at least one of and preferably all of Ras Homologue gene family member AB (RhoA/B), Rho Associated coiled-coil kinase (ROCK), and LIM-motif containing kinase (LIMK2). Activation of the pathway induced signaling and actin reorganization. The pathway is not activated in LNCAP cells. Testosterone-BSA induced apoptosis in both LNCAP and DU145 cells, an effect regulated by the actin cytoskeleton reorganization. In addition, in vivo experiments in LNCaP-inoculated nude mice revealed that treatment with testosterone-BSA (8 mg/kg weight) for one month resulted in a 60% reduction of tumor-size, compared to control animals. This effect was not observed with the anti-androgen flutamide. No apparent toxic effects were seen in control animals. The Examples show that activation of the mAR can induce apoptotic regression of prostate cancer cells.

Accordingly, and in one embodiment of the invention, one or a combination of the foregoing agent detection methods can further include the step of detecting modulation of a signaling molecule downstream of the mAR. By “downstream” is meant that the particular molecule is in a biochemical pathway that is responsive to mAR activation. Preferably, such a method further includes the step of detecting a change in the level of the signaling molecule, relative to a suitable control (e.g., one in which agent is not added). The step of detecting modulation of the signaling molecule can include, for instance, measuring activity (ie., an increase or decrease) of at least one of guanine nucleotide binding or exchange, kinase activity, guanosine triphosphatase activity, phosphatidylinosotol breakdown, diacylglycerol, inositol triphosphate, actin reorganization, reporter gene expression, and intracellular calcium level. Preferably, the kinase is one of focal adhesion kinase (FAK) or phosphatidylinositol-3 (PI-3) kinase. Such method can further include the step of detecting association of FAK with phosphatidylinositol-3 (PI-3) kinase. A particular guanosine triphosphatase is Cdc42/Rac1.

Immunological methods for detecting certain of these and other signaling molecules have been reported. See Papakonstanti, E. et al. (2003), supra; and Papadopoulou, N. D. et al. (2004); supra. Generally, the methods include established immunoprecipitation, kinase, and immunoblotting type detection formats.

### C. Modulation of Intracellular Calcium By mAR.

It has been reported that a common effect of testosterone binding to the mAR is a rapid, within seconds, increase in intracellular calcium. See Kampa, et al. (2004), supra; and Kampa and Castanas (2006), supra. The increase has been reported to be sensitive to the G protein receptor antagonist, pertussis toxin indicating a G-protein coupled receptor. It has also been seen in cells lacking the AR and was not effected by antiandrogens such as cyproterone acetate or flutamide. This indicates, among other things, a non-classical receptor mediated action. See also Gong et al. (1995) Endrocrinology 136: 2172.

Accordingly, and in one embodiment, one or a combination of the foregoing methods for detecting mAR modulation can be combined with a conventional test for detecting and optionally quantifying an increase in intracellular calcium. Examples of suitable tests have been described, for instance, by Kampa et al. (2004), supra. Additionally suitable tests will generally employ at least one of a calcium probe, an antibody to a calcium probe, a calcium ionophore and a calcium chelator to detect changes in intracellular calcium. See the Sigma-Aldrich Catalogue Book entitled Cell Signaling & Neuroscience (2002-2003), particularly pages 482-498, for disclosure related to the use of such materials to detect and measure calcium levels. Exemplary formats include use of Arsenazo III, Fluo 3, Fluo-3 am, Fura2 and as detectable indicators of an increase (or decrease) in intracellular calcium.

Thus in one embodiment, the intracellular calcium level of suitable cells, cell lines or tissue samples (eg., DU-145 or LnCaP cells) is detected following contact of the mAR with one or more agents. Any increase in the level of intracellular calcium, relative to a suitable control such as incubutation without agent, can be taken to be indicative of an agent that modulates the mAR. Preferred agents will increase the level of intracellular calcium, as measured by one or a combination of the foregoing standard assays, by at least about 20%, preferably at least about 50%, and more preferably at least about 90%, 95%, 100% up to about 300%, 500%, up to about 1000%, relative to the control without agent.

### D. Modulation of Prostate Specific Antigen (PSA)

It has been reported that stimulation of the mAR is accompanied by secretion of PSA. See Kampa, M et al. (2002), supra. In particular, the secretion was inhibited after pretreatment with cytochalasin B. Thus, one or a combination of the foregoing methods for detecting an mAR modulating agent can further include a test for detecting a change in the level of PSA secreted by the cells, cell line or tissue used in the assay. In one embodiment, such a test further includes the step of detecting any PSA expressed by the cells, cell line, or tissue samples, relative to a suitable control (eg., no addition of the agent). Suitable cells, cell lines and tissue samples can be known or suspected of expressing the mAR, iAR or both.

A particular method for detecting the PSA relies on convention immunological approaches. In one embodiment, the PSA is measured by contacting secreted PSA with an anti-PSA monoclonal antibody that binds PSA specifically. Such an assay can be performed in any number of acceptable formats such as by using direct detection or sandwich assay formats. A variety of suitable antibodies that have been reported to be reactive against PSA can be obtained from the ATCC.

zzparticular agents of interest according to above-mentioned PSA assay will increase the level of PSA secretion, by at least about 20%, preferably at least about 50%, more preferably at least about 90%, 95%, 100% up to about 200% or 300%, relative to a suitable control assay without agent. Preferably, the PSA is detected using one or a combination of suitable immunological reagents such as mono- or polyclonal antibodies that are known to bind PSA.

E. Modulation of Apoptosis By Membrane Androgen Receptor (mAR)

It has been reported that activation of the mAR results in apoptotic regression of prostate cancer cells both in vitro and in vivo. Hatzoglou, A. et al. (2005); supra. Thus, one or a combination of the foregoing methods for detecting modulation of the mAR can further include the step of detecting a change in the level of apoptosis in the cells, cell line or tissues used in the assay. Accordingly, and in one embodiment, the method for detecting agent further includes the step of detecting cell death (apoptosis) in the cells, cell lines, or tissue samples that express the mAR. Preferably, an agent of the invention increases the level of apoptosis in the cells, cell line and tissues used in the assay, relative to a suitable control (eg., performing the assay in the absence of agent).

A variety of suitable assays for detecting and quantifying apoptosis has been reported including immunological, dye mediated and cytometric assays. See Hatzoglou, A. et al. (2005); supra. Other suitable assays have been disclosed, for instance, by the Sigma-Aldrich Catalogue Book entitled Cell Signaling & Neuroscience (2002-2003), particularly pages 17-98. Thus in one invention embodiment, the apoptosis is detected by assaying at least one of an annexin, a detectable small molecule, and an apoptotic or pro-apoptotic protein. Examples include BCL-2, BCL-X, Caspase 9, Apaf-1 among others.

Preferred practice of the invention involves performing one or more of the contacting steps with a desired cell, cell line, or tissue sample that is known or suspected of expressing the mAR. Thus in one embodiment, the invention encompasses a method for detecting a suitable mAR receptor ligand in which DU-145 cells, for instance, are used as a source of mAR. Alternatively, LNCaP or prostate gland tissue is used as the source of the mAR. The mAR can be provided, depending on the assay format selected, as whole cells or tissues or as a subcellular fraction (usually a membrane fraction).

Particular agents of interest according to above-mentioned apoptosis assay will increase the level of cell death, by at least about 20%, preferably at least about 50%, more preferably at least about 90%, 95%, 100% up to about 200% or 300%, relative to a suitable control assay such as one in which no agent is used.

The following paragraphs F-G describe exemplary methods for detecting and quantifying the capacity of an agent or agents (e.g., a library of agents) to modulate (increase or decrease) the AR.

### F. Control Assays for Detecting AR Modulation (In Vitro)

As discussed, in many invention embodiments it will be useful to perform one or a combination of the control assays to detect and optionally quantify the capacity of an agent to increase or decrease AR function. In one embodiment, a suitable AR-expressing cell, cell line or tissue sample is contacted with agent to identify such activity, if any. The AR has been reported to be expressed in numerous body tissues such as male reproductive tissue (including the prostate gland and accessory tissues), female productive tissue, certain CNS tissues as well as certain bone and muscle cells. The AR is believed to be generally sensitive to the endogenous androgen ligands testosterone and 5-α-dihydrotestosterone. There is recognition that the AR is composed of three functional domains: a ligand binding domain, the DNA-binding domain, and an amino-terminal domain. See, for example, Caplan, et al. (1995) J. Biol. Chem. 270: 5251; Fliss et al. (1999) J. Biol. Chem. 274: 34045; and Fang et al. (1996) J. Biol. Chem. 271:28697; and references cited therein.

An agent that associates with the AR, for instance an AR receptor ligand and mimics the effect of a natural AR ligand is referred to herein as an “AR agonist” while a compound that inhibits the effects of a natural ligand is called an “AR antagonist”. Preferred agents according to the invention have negligible or no activity as an AR agonist or antagonist as determined by any one or combination of the control assays disclosed herein.

Various cells lines are available from the ATCC (Manassas, Va. (USA)) that are reported to express the AR eg., RBA, TRAMP-C2, DDT1 MF-2, RWPE-2, and others. MDA (DA-MB-453) cells have also been reported to express an endogenous AR. See PCT/US2005/029592 and references disclosed therein. MDA cells are particularly preferred in embodiments in which the control assay is a radioligand displacement assay of the affinity of agent for the MDA AR. See PCT/US2005/029592 at pgs. 54-56. Another cell line that express the AR is LNCaP.

A recombinant human androgen receptor (hAR) can be used in certain control assays. In one embodiment, a competitive binding analysis is performed on cells that express the hAR. Such cells are known and include for instance, baculovirus/Sf9 generated hAR cells and extracts thereof. Alternatively, other cells, cell lines and tissues that are known to express the AR can be used, for instance, MDA cells. See Sambrook et al. in Molecular Cloning: A Laboratory Manual (2d ed. 1989); and Ausubel et al. (1989), Current Protocols in Molecular Biology, John Wiley & Sons, New York for a discussion relating to performing standard recombinant techniques.

Once a suitable cell, cell line or tissue that expresses the AR (or hAR) is obtained, binding is performed in the presence or absence of differing concentrations of agent and a fixed concentration of ³H-DHT tracer. See Chang, et al. (1984) J. Steroid Biochem. 20: 11 for general disclosure relating to the assay. Briefly, progressively decreasing concentrations of agents are incubated in the presence and absence of the AR or hAR extract, for example, along with hydroxyapitite, and about 1 nM ³H-DHT for about one hour at 4 C. Subsequently, binding reactions are washed to remove excess label. AR or hAR bound ³H-DHT levels are determined in the presence of compounds (competitive binding) and compared to binding without agent (maximum binding). See Chang et al. (1992) PNAS 89: 5546. Agent binding affinity to the hAR is expressed as a concentration of the agent at which one half of the maximum binding is inhibited (IC₅₀).

Various nucleic acids such as cDNA that encode the human androgen receptor have been reported. See Chang, C S. (1988) Science 240: 324. See also the National Library of Medicine (GENBANK) for other suitable sequences, particularly those referenced under accession no. NM_{—}001011645.

Particular agents in accord with the invention show negligible or no activity in these assays. Preferably, such agents exhibit an IC₅₀ in excess of about 10 to about 1000 micromolar or more.

i. Detection of Androgen Response Element (ARE) Activation

In some control embodiments, it will be useful to express a heterologous AR (eg., hAR) in cells that do not normally express the receptor (eg., COS or CV-1 cells). Preferably, the AR is transiently expressed by employing recombinant nucleic acid techniques so that the hAR is expressed. In one approach, cells are cultured under conditions permitting expression of the heterologous AR and contacted by compounds with potential to modulate the AR. Modulation of the AR is monitored approaches that can detect changes in the activity of a transfected plasmid construct that includes a detectable androgen responsive element (ARE). Illustrative AREs include an androgen responsive gene promoter, androgen responsive gene enhancer, and the like. An agent that binds the AR can be detected in the method by monitoring “downstream” ARE activity.

In one approach outlined in PCT/US2004/027483 (WO 2005/018573), suitable cells (COS or CV-1) are transfected in accord with materials and methods disclosed by Berger et al., J. Steroid Biochem. Mol. Biol. (1992) 41: 733. Constructs to be used include pShAR (expresses AR), MTV-LUC, pRS-β-GAL and pGEM (filler DNA). The receptor plasmid, pRShAR, contains the human AR under constitutive control of the SV-40 promoter. See Simental et al. J. Biol. Chem. (1991) 266:510. The reporter gene construct, MTV-LUC, contains a cDNA encoding firefly luciferase (LUC) under the control of the mouse mammary tumor virus (MTV) long terminal repeat, a conditional promoter that includes an androgen response element. See Berger, et al., supra. Additionally, pRSβ-GAL coding for constitutive expression of E. coli β-galactosidase can be included as an internal control.

Following transfection, media is removed from the transfected cells and the cells are washed. Media containing an agent according to the invention is then added to the cells. Any acceptable agent concentration can be used such as one ranging from 10⁻¹² M to 10⁻⁵ M. After incubating the cells in the media for about 2-3 days, preferably about 2 days, the cells are washed, lysed with Triton X-100 or another suitable detergent and assayed for LUC and β-GAL activities using standard procedures. Further disclosure regarding this particular assay, including use of positive controls for a AR antagonist (2-OH-flutamide) and AR agonist (DHT) can be found in the PCT/US2004/027483 patent application.

Reference herein to an “MTV-ARE assay” refers to the assay disclosed in the PCT/US2004/027483 application. Preferred agents in accord with the invention will show negligible or no activity in the MTV-ARE assay when compared to one or more suitable controls (eg., relative to the activity of DHT in the assay).

See also Panomic, Inc. (6519 Dumbarton Circle, Fremont Calif. (USA)) for a list of reporter gene constructs (vectors) that can also be used to detect and optionally quantify ARE activity (e.g., pAR-LUC is one such vector). Instructions for using such vectors can be obtained from the company.

### G. Control Assays for Detecting AR Modulation (In Vivo)

For some invention applications, it will be useful to investigate potential for an agent to modulate the AR in vivo. Significantly, use of multiple detection assays (e.g., a combination of the in vitro and/or in vivo assays) with a single agent (or even a library of agents) can extend the selectivity and sensitivity of detection.

Such broad spectrum testing provides additional advantages. Thus, for example, in vitro assays of the invention can efficiently perform multiple analyses, thereby increasing efficiency and probability of identifying agents with potential therapeutic capacity. This is especially useful when large numbers of agents need to be tested. For instance, libraries of potential mAR binding ligands and other agents can be made by standard synthetic methods including combinatorial-type chemistry manipulations and then tested in accord with the invention.

Thus in one embodiment, one or a combination of the foregoing methods further includes the step of detecting any activation of the iAR in in vivo. Suitable methods for detecting modulation of the AR have been described. See, for example, one or more of PCT/IB2005/002592 (WO 2006/024931); PCT/US2004/038859 (WO 2005/052118); PCT/2004/027483 (WO 2005/018573); PCT/2005/013775 (WO 2005/105091); PCT/US2005/029592 (WO 2006/026196); and PCT/IB2005/002592 (WO 2006/024931); and references disclosed therein.

In a particular embodiment, agents of the invention are tested in the in vivo prostate assay disclosed by PCT/US2005/029592. Briefly, young sexually mature rats are subjected to an orchiectomy (ORX). A dose of the agent is administered sub-cutaneously or orally to the rat, for instance between from about 0.1 micrograms/kg to about 100 micrograms/kg, about 1-2 times daily for about one to two weeks. After this period, the ventral portion of the prostate gland and seminal vesicles are each weighed. The extent to which agents inhibit ORX-induced loss of these tissues is assessed by standard methods.

Thus in one embodiment, the foregoing in vivo method further includes selecting an agent with at least one of the following properties, relative to a suitable control, in the test mammal: (1) negligible or no increase in prostate gland weight; (2) negligible or no increase in body weight; (3) negligible or no decrease in body fat; and (4) negligible or no increase in bone density. In this invention example, the method can further include selecting an agent that has at least one of the foregoing properties as being further indicative of presence of the agent.

Reference herein to the detection of “negligible or no” signal or activity, when used to describe one or a combination of results in a suitable control, means less than about 20% of the activity of DHT in that assay, preferably less than about 10% of that activity, more preferably less than bout 5%, less than about 3% or less than about 1% of the activity of DHT in the assay. A preferred agent of the invention will exhibit negligible or no capacity to modulate the AR according one or a combination of the control assays described above.

A highly preferred agent according to the invention is, for instance, an mAR receptor ligand that exhibits no detectable activity in one or more of the control assays described in Parts F and G. In contrast, such a preferred agent will exhibit substantial activity in one or more of the assays provided in Parts A-E, above.

Optimal practice of a particular control assay will sometimes require that at least one of the mAR and iAR be provided in a fractionated, synthetic or semi-synthetic format. For instance, and in one embodiment, the iAR is present as a synthetic liposome composition. Method for producing such compositions have been reported. Other receptor embodiments include presentation on virus-induced budding membranes containing the iAR. See WO0102551 and Mirzabekov et al., 2000.

The present invention is flexible and can be used in a variety of assay formats. Accordingly in one embodiment for testing an agent for capacity to modulate the mAR, a subcellular fraction from cells, a cell line, or a tissue sample that are known or suspected to express the mAR and/or iAR. Thus in one embodiment, a control assay as described herein can be performed using a subcellular fraction of cells that express the iAR, preferably a membrane, cytoplasmic or nuclear fraction.

One or a combination of assays for detecting mAR and iAR modulation can be performed using one or more of the following detecting steps: label displacement, surface plasmon resonance, fluorescence resonance energy transfer, fluorescence quenching, fluorescence polarization, luminescence, chemiluminescence, fluorescence, absorption, and scintillation counting. Choice of a particular detection format will depend on conventional variables such as the detectably-labeled androgen selected and the degree of assay sensitivity required, for instance.

### H. Agent Sources

The present invention can be used to detect and optionally identify a wide variety of agents. Preferred agents include a peptide, peptoid, a polypeptide, an antibody or antigen-binding fragment thereof, a lipid, a carbohydrate, a nucleic acid, a small molecule; or a combination thereof. As used herein, the term “small molecule” refers to a compound having molecular mass of less than 3000 Daltons, preferably less than 2000 or 1500, still more preferably less than 1000, and most preferably less than 600 Daltons. A “small organic molecule” is a small molecule that comprises carbon.

A wide variety of collections are available from which to select agents according to the invention. Particular agents for testing can be included within large libraries of synthetic or natural compounds. Numerous means are currently used for random and directed synthesis of saccharide, peptide, and nucleic acid based compounds. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are available and can be prepared. Alternatively, libraries of natural compounds in the form of bacterial, fungal, plant and animal extracts are available from e.g., Pan Laboratories (Bothell, Wash.) or MycoSearch (NC), or are readily producible by methods well known in the art. Additionally, natural and synthetically produced libraries and compounds are readily modified through conventional chemical, physical, and biochemical means.

Other sources of potential agents include a variety of monomeric and oligomeric flavanols, particularly catechin, epicatechin and derivatives thereof. See Nifli, A-P et al. (2005) Exp. Cell Res. 309: 329. Additional sources of potential agents include certain opioids. See Kampa, M. et al. (2004) Exp. Cell Res. 294: 234.

Alternatively, one or a combination of small molecules said to bind the AR can be tested for preferential mAR binding. See eg., PCT/IB2005/002592 (WO 2006/024931); PCT/US2004/038859 (WO 2005/052118); PCT/2004/027483 (WO 2005/018573); PCT/2005/013775 (WO 2005/105091); PCT/US2005/029592 (WO 2006/026196); and PCT/IB2005/002592 (WO 2006/024931).

Useful agents in accord with the invention may be found within numerous chemical classes. Useful compounds may be organic compounds, or small organic compounds. Small organic compounds have a molecular weight of more than 50 yet less than about 2,500 daltons, preferably less than about 750, more preferably less than about 350 daltons. Exemplary classes include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

Additional agents that can be tested in accord with the invention include a serum albumin molecule bound to one or more of the carbon atoms of the testosterone ring, eg., the 3′ carbon of testosterone (eg., T-3-BSA), or 17′ carbon of testosterone (e.g., T-17-BSA). In one embodiment, the serum album is spaced from the steroid ring by a suitable linker such as an oxime linker bound to testosterone at the 3′ carbon. See Goeij, A F et al. (1986) J. Steroid Biochem. 24: 1017. It will be appreciated that other compounds can be useful agents such as testosterone bound to a fragment of a serum albumin (BSA or human serum albumin (HSA)). Preferred fragments include those sequences that have at least 50%, preferably at least about 70%, more preferably at least about 90%, about 95% up to about 99% of a serum albumin amino acid sequence. Suitable fragments can have consecutive or non-consecutive deletions.

By “bovine serum albumin” or BSA is meant the amino acid sequence represented by Swiss-Prot entry P02769 including preferred fragments and allelic variants thereof. By “human serum albumin” or HSA is meant the amino acid sequence represented by Swiss-Prot entry P02768 including preferred fragments and allelic variants thereof. Thus, an allelic variant of the aforementioned serum albumin sequences can be used to make an agent of the invention (preferred fragments of such variants are encompassed as well).

For primary screening, a useful concentration of an agent to be tested is from about 10 micromolar to about 100 micromolar more (i.e. 1 mM, 10 mM, 100 mM, or even 1M), but can also be 1 nM and higher, 1 pM and higher, or 1 fM and higher. The primary screening concentration will be used as an upper limit, along with nine additional concentrations, wherein the additional concentrations are determined by reducing the primary screening concentration at half-log intervals (e.g. for 9 more concentrations) for secondary screens or for generating concentration curves.

The terms “protein” and “polypeptide” may be used interchangeably and mean at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U138 Part 2 Aug. 22, 1999, both of which are expressly incorporated by reference herein.

As used herein, the term “detectable step”, “detectable” and the like refers to a step that can be measured, either directly, e.g., by measurement of a second messenger or detection of a modified (e.g., phosphorylated) protein, or indirectly, e.g., by monitoring a downstream effect of that step. For example, adenylate cyclase activation results in the generation of cAMP. The activity of adenylate cyclase can be measured directly, e.g., by an assay that monitors the production of cAMP in the assay, or indirectly, by measurement of actual levels of cAMP. Other detectable steps include the measurement of calcium flux, apoptosis, PSA secretion and reorganization of the cytoskeleton.

As used herein, the terms “change”, “difference”, “decrease”, or “increase” as applied to e.g., binding or signaling activity, or PSA secreation, apoptosis and/or calcium flux refer to an at least 10% increase or decrease in each of those activities.

### G. High Throughput Screening Assays

As discussed, it is within the scope of the present invention to use one or more detectably-labeled androgens to: (1) detect cells that express teh mAR; and (2) screen for mAR binding receptor ligands and other agents. Such a screen can be readily adapted so that it can be performed in a high- or ultra-high throughput format. Practice of this invention embodiment can be achieved by using a fluorescent, luminescent, phosphorescent, or chemiluminescent compound(s) to label the androgen either directly or indirectly, as described previously.

When using fluorescently labeled androgens, one or a combination of different fluorescent monitoring systems can be used. These include, for instance, FACS systems. Preferably, FACS systems are used or systems dedicated to high throughput screening, e.g 96 well or greater microtiter plates. Methods of performing assays on fluorescent materials are well known and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park Benjamin/Cummings Publishing Col, Inc. (1978), pp. 296-361. A variety FACS systems are known in the art and can be used in the methods of the invention (see e.g., WO99/54494, filed Apr. 16, 1999; U.S. application No. 20,010,006,787, filed Jul. 5, 2001, each expressly incorporated herein by reference).

For instance, a substantial loss in fluorescently labeled androgen from mAR positive cells in the presence of agent is indicative of agent with potential to bind the mAR. Such a method result can readily be achieved using standard high- and ultra-high throughput screening approaches. If desired, such an approach can be combined (eg., run essentially in parallel with) with one or more of the suitable control assays disclosed herein which controls may also be conducted in a high- or ultra-high throughput platform.

According to one invention embodiment, fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation which has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation.

In another embodiment, flow cytometry is used to detect fluorescence. Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference).

See also Olah, M M et al. (2004) Current Drug Discov. Technology, 3: 211; deLong et al. (2005) in J. Chromatogr. B. Analyt. Biomed. Life Science, 829: 1; and Liu B. et al. (2005) Am. J. Pharmacogenomics 4: 263 for general disclosure relating to performing high throughput and related screening assays.

Unless otherwise specified, reference herein to a testosterone-BSA conjugate (including various abbreviated forms) means the following particular compound: testosterone 3-(O-carboxymethyl)oxime:BSA.

All documents mentioned herein are incorporated by reference in their entirety. The following non-limiting examples are illustrative of the invention.

### REFERENCE EXAMPLE

The present reference example is reproduced from Kampa, M et al. (2002) FASEB J. 16:1429. It shows, among other things, saturation and displacement binding of tritiated-testosterone on membranes of cells that express the mAR (LNaCP) cells.

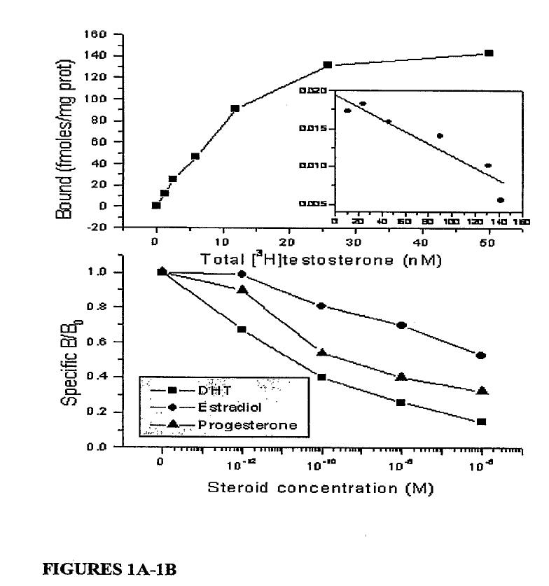

Turning to FIGS. 1A-B, it shows saturation (1A) and displacement (1B) binding of ³H-testosterone on membranes of LNCaP cells. (A) saturation binding. Cell membranes were prepared in accordance with Kampa, M. et al. (2002), ibid, at a final concentration of 2 mg/ml. They were incubated overnight at 4 C with six concentrations of ³H-testosterone, which varied from 2 to 50 nM in the absence or in the presence of a thousand-fold excess of unlabeled androgen (DHT). The figure presents the analysis of the data in Scatchard coordinates. The figure represents the results of a typical experiment in triplicate. (1B) Displacement binding: Cell membranes (200 micrograms) were incubated with about 5 nM of ³H-testosterone alone (Bo) or in the presence of the indicated concentrations of unlabeled steroids (DHT, estradiol, progesterone), ranging from 10⁻¹² to 10⁻⁶M. Nonspecific binding was assayed by introducing 5 micromolar DHT. The figure (means of three different experiments performed in duplicate) presents the ratio of specific binding in the presence of the indicated concentrations of DHT (Bs) to the specific binding in the absence of DHT (Bo), Bs/Bo.

There have been reports of the identification of functional mARs for androgens in a prostate cancer cell line (Kampa et al 2002), as well as in human prostate tumors, been highly expressed on cancer cells (Stathopoulos et al 2003) Activation of these binding sites by testosterone-BSA, a no permeable analog of testosterone was shown to induce rapid actin cytoskeleton reorganization and to increase within minutes the secretion of prostate-specific antigen (PSA). A specific non-genomic signaling cascade regulating the molecular mechanism of actin reorganization was identified (Papakonstanti et al 2003). In addition, in a more recent study BSA-coupled testosterone was shown to induce a) apoptosis of either iAR-negative DU145 or iAR-positive LNCaP human prostate cancer cells and b) regression of prostate cancer cells both, in vitro and in vivo (Hatzoglou et al 2005). These findings imply that activators of mAR are important tools for the apoptotic regression of prostate cancer. However, the molecular mechanism controlling the observed actin redistribution and the induction of apoptosis is not completely understood.

As shown below, the iAR-negative DU145 prostate cancer cell line was used and shown to express active mAR (Hatzoglou et al 2005), to explore in detail the cross talk between actin cytoskeleton reorganization and apoptosis. Molecular mechanisms regulating the actin reorganization and the induction of the apoptotic response of DU145 cells upon Testosterone-BSA treatment were addressed and indicate a central regulatory role of actin cytoskeleton rearrangements in the apoptotic regression of prostate cancer cells.

### Example 1

### Testosterone-BSA Induces Rapidly Potent Actin Polymerization in DU145 Cells

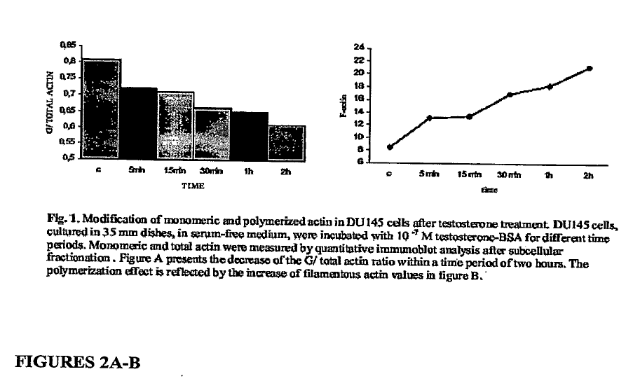

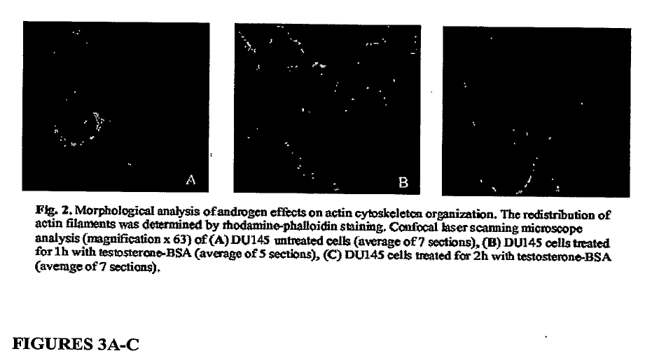

There has been recognition that activation of mAR sites in LNCaP human prostate cancer cells results within minutes, in a significant rearrangement of the actin cytoskeleton, as indicated by the potent actin polymerization and microfilament reorganization (Kampa et al 2002). Using both, non-permeable testosterone analogs and specific inhibitors or antagonists of the intracellular androgen receptors (iAR), we provided strong evidence that in LNCaP cells this phenomenon was induced through the activation of the mARs. In order to provide ultimate experimental evidence for the cross-talk between mAR activation and actin cytoskeleton in humane prostate cancer cells, we explored whether testosterone-BSA may affect actin cytoskeleton dynamics in the iAR-negative but mAR-positive DU145 cells. As calculated from FIG. 2A-B, incubation of cells with 10⁻⁷M testosterone-BSA conjugate resulted in a significant decrease of Triton-soluble (monomeric) to Triton-insoluble (filamentous) actin ratio by 45%. These quantitative results were fully supported by confocal laser scanning microscopic analysis of the actin cytoskeleton in testo-BSA treated DU145 cells. Indeed, a complete submembranous redistribution of the microfilamentous structures became evident within 10 minutes and persisted for at least 2 hours, as shown in FIGS. 3A-C. From these results we can conclude that activation of mARs by androgens induce rapid and potent actin cytoskeleton redistribution in prostate cancer cell, independently of the expression or not of intracellular androgen receptors.

### Example 2

### Actin Reorganization is Required for the Testosterone-BSA Induced Apoptotic Response of DU145 Cells

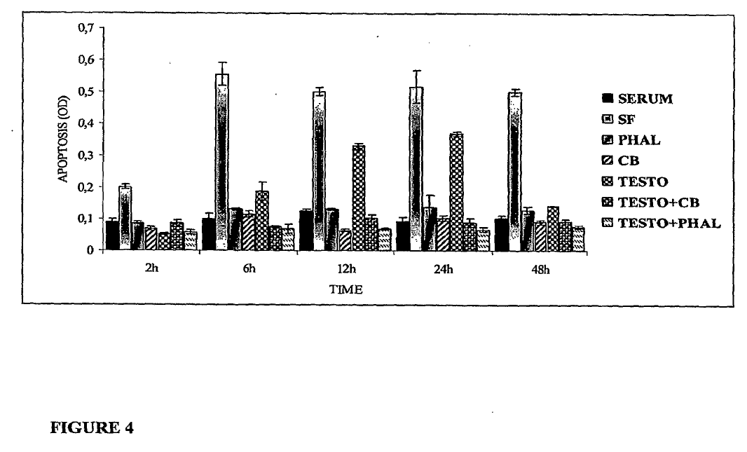

There are reports that activation of mAR by Testo-BSA resulted in a significant induction of apoptosis in both, LNCaP and DU145 prostate cancer cell lines. In order to analyze a possible cross-talk between actin cytoskeleton reorganization and apoptosis upon mABS activation, we studied the apoptotic response of DU145 cells in the presence of specific agents blocking actin reorganization. Incubation of the cells with cytochalasin B (10⁻⁶ M) or phallacidin (10⁻⁶ M), which prevent actin redistribution showed a clear inhibition of the apoptotic response of DU145 cells treated with Testo-BSA (FIG. 4). The FACS analysis fully supported these observations. From these results it is concluded that actin redistribution is a necessary step, which may regulate the signaling events leading to the apoptotic response of DU145 prostate cancer cell upon activation of the testosterone membrane binding sites. This assumption is in line with previously published data, showing that actin reorganization is an important signaling step, regulating NFkB translocation and transcriptional regulation of TNFα induced anti-apoptotic response (Papakonstanti and Stounaras, Mol Biol Cell, 2004).

### Example 3

### Activation of Mars in DU145 Cells Fails to Induce the Fak/PI-3K/Rac1 Pathway

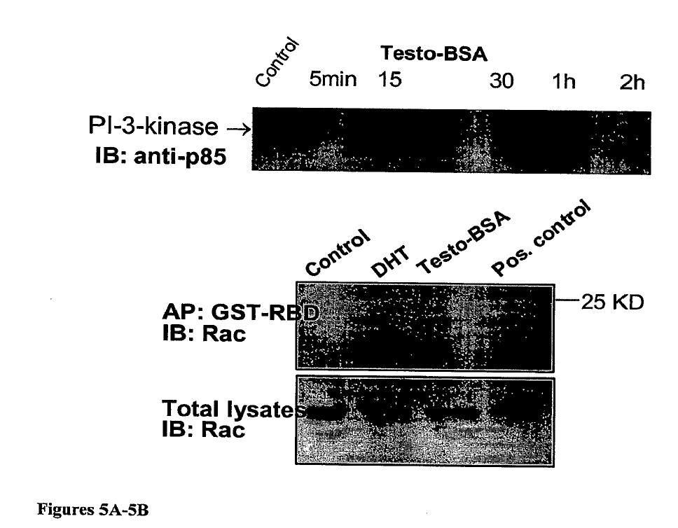

Activation of mAR in LNCaP cells was shown to induce a rapid non-genomic signaling pathway involving FAK/PI-3K/Cdc42/Rac1 and leading to actin reorganization (Papakonstanti et al. Mol Endocrinol 2003). Surprisingly, in iAR-negative DU145 cells this pathway does not seem to be involved in mediating the rapid, non-genomic actin signaling. Indeed, as shown in FIGS. 5A-B, FAK was constitutively phosphorylated in DU145 cells. When incubating the cells with testo-BSA, neither apparent additional FAK phosphorylation could be documented, nor activation of the downstream signaling molecules PI-3K, and Rac1. In line with these results, incubation of DU145 cells with the specific PI-3K inhibitor wortmannin had no effect on actin polymerization induced by testosterone treatment, indicating that PI-3K signaling is not involved in testosterone-induced rapid actin reorganization in these cells. These findings imply the existence of an alternative non-genomic signaling cascade regulating the rapid actin reorganization shown in FIGS. 2A-B.

### Example 4

### Activation of Mars in DU145 Cells Induces Rapid RhoA and ROCK Activation

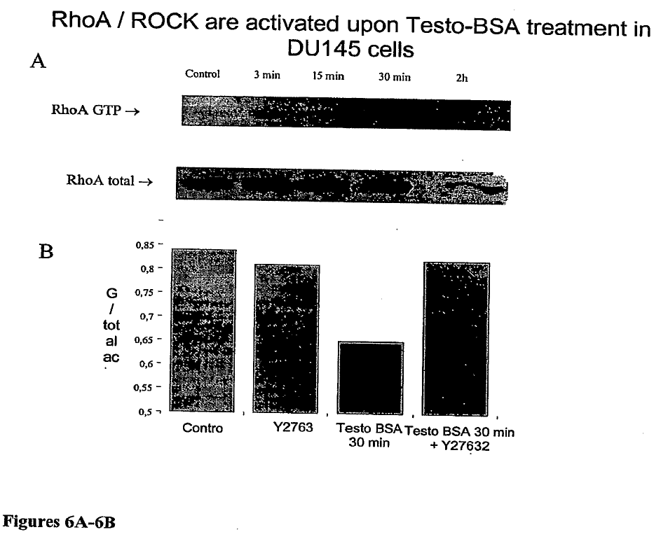

Looking for an alternative mechanism mediating the non-genomic testosterone signaling in DU145 cells we analyzed a possible rapid activation of Rho-GTPase's. Indeed, among the signaling effectors that regulate such processes of actin reorganization, the best understood are small GTPases of the Rho family. Classically, plasma membrane receptors activate specific guanine exchange factors (GEFs) often via phosphorylation, which leads to subsequent loading of Rho GTPases with GTP. Active Rho GTPases continue signal transmission towards Rho coiled-coiled kinase 1 (ROCK1) and/or p21-activated kinase 1 (Pak1). Activated Pak1 and ROCK1 phosphorylate and activate Lim-kinases 1 and 2 (Limk1 and Limk2) respectively. Limk1 and Limk2 are closely related proteins composed of two N-terminal Lim domains and a C-terminal kinase domain. The Lim domains are protein-binding motifs frequently found in cytosolic proteins that interact with the actin cytoskeleton. Eventually, Limk1 and Limk2 phosphorylate actin-depolymerizing proteins such as cofilin and actin-depolymerizing factor (ADF), which are inactivated and thus permit actin polymerization to occur. As shown in FIG. 6A-B, treatment of DU145 cells with 10-7 M testosterone-BSA induced a very fast (within 3 minutes) activation of RhoA, which persisted for up to two hours with a maximum activation after 30 minutes. To further analyze the downstream effector of RhaA activation, DU145 cells, activated with testo-BSA were treated with the specific ROCK inhibitor Y27632 and actin redistribution was documented quantitatively using the G/Total-actin ratio. As shown in FIG. 6B, the testosterone induced rapid actin polymerization was totally inhibited in the presence of Y27632, indicating that ROCK is a RhoA effector in the non-genomic signaling of mAR activation in DU145 cells.

### Example 5

### Inhibition of the Rho/ROCK Signaling Cascade Prevents Both Actin Reorganization and Induction of Apoptosis

As indicated in FIG. 4, actin redistribution seems to be a necessary step, controlling the signaling events leading to the apoptotic response of DU145 prostate cancer cell upon activation of the mARs. To further study this phenomenon, we analyzed the apoptotic response of DU145 cells in the presence of inhibitors blocking the newly identified signaling cascade, leading to actin reorganization. Interestingly, incubation of DU145 cells with Y27632 resulted in abrogating the pro-apoptotic cell response upon testo-BSA treatment, as shown in FIGS. 6A-B. Taken together the results of the figures indicate that inhibition of the Rho/ROCK signaling cascade prevents both, actin reorganization and induction of apoptosis. These results support the hypothesis that actin redistribution is a key regulatory player in controlling the apoptotic response of mABS-activated DU145 cells. On the other hand, incubation of the cells with wortmannin had no effect on testo-BSA induced apoptotic response, supporting the evidence that PI-3K signaling is not involved in apoptotic cell response of the iAr-negative DU145 cells.

The following materials, methods, and scientific references will aid in the understanding of invention.

### Materials

RPMI 1640 and fetal bovine serum (FBS) were from Gibco-BRL (MD, USA). Rhodamine-phalloidin and Slow Fade Antifade kit were from Molecular Probes, Inc. (Eugene, Oreg.).

The ECL Western blot kit was obtained from Amersham (Arlington Heights, Ill., USA). Testosterone-3-(O-carboxymethyl)oxime-BSA (named testosterone-BSA) was obtained from Sigma (St. Louis, Mo.). Testosterone-BSA was dissolved in culture medium at ten times the concentration indicated in each experiment. The stock solution was then treated with a solution of charcoal (30 mg/ml) and dextran (0.03 mg/ml) for 30 min at RT, centrifuged at 3000×g for 15 min and passed through a 0.22 μm filter, to remove any potential contamination with free steroid. The treated testosterone-BSA was subsequently used throughout all studies. All other chemicals were obtained from usual commercial sources at the purest grade available.

### Cells and Culture Conditions

DU145 human prostate cancer cells isolated from a metastatic brain carcinoma (DMSZ, Braunschweig, Germany) were studied between passages 2 and 9. Cells were cultured in RPMI 1640 medium supplemented with 10% heat-inactivated fetal bovine serum at 37 C in a humidified atmosphere of 5% CO₂ in air. They were subcultured once a week and incubated in serum-free medium for 24 h before any experiment.

### Immunoblot Analysis of Soluble and Insoluble Actin

After treatment with 10⁻⁷ M testosterone-BSA, cells were incubated in 0.5 ml of Triton extraction buffer (0.3% Triton X-100, 5 mM Tris-HCl, 2 mM EGTA, 300 mM sucrose, 400 μM PMSF, 10 μM leupeptin, 2 μM phalloidin, pH 7.4) for 5 min on ice. The Triton-soluble proteins were precipitated with equal amounts of 6% perchloric acid (PCA). The Triton-insoluble fractions were precipitated with 1 ml of 3% PCA. Equal volumes of each fraction were subjected to SDS electrophoresis and proteins were transferred to a nitrocellulose membrane, using the LKB electrophoresis apparatus (LKB, Bromma, Sweden). Membranes were then incubated with monoclonal anti-actin antibody, followed by incubation with the appropriate labeled secondary antibody. Blots were developed using the enhanced chemiluminescence system, and the band intensities were quantitated by PC-based image analysis (Image Analysis, Inc., Ontario, Canada).

### Confocal Laser Scanning Microscopy

Direct fluorescence microscopy of filamentous (F)-actin was performed by rhodamine-phalloidin staining of DU145 cells treated with 10⁻⁷ M testosterone-BSA for 1 h and 2 h respectively. The samples were analyzed with a confocal laser scanning module (Leica Lasertechnik, Heidelberg, Germany).

### Quantitative Measurement of Apoptosis

The APOPercentage Apoptosis Assay (Biocolor Ltd., Belfast, N. Ireland) was used to quantify apoptosis, according to manufacturer's instructions. This kit uses a dye that stains red the apoptotic cells undergoing the membrane “flip-flop” event when phosphatidylserine is translocated to the outer leaflet. Detection of apoptosis can be readily observed under inverted microscopy. For apoptosis quantitation, the amount of dye within the labeled cells can subsequently be released into solution and the concentration measured at a wavelength of 550 nm (reference filter 620 nm) using a color filter microplate calorimeter (Dynatech MicroElisa reader Chantilly, Va.).

See also Etienne-Manneville, S., and Hall, A. (2002) Nature 420(6916), 629-35; Schmidt, A., and Hall, A. (2002) Genes Dev 16(13), 1587-609; Amano, M., Fukata, Y., and Kaibuchi, K. (2000) Exp Cell Res 261(1), 44-51; Jaffer, Z. M., and Chernoff, J. (2002) Int J Biochem Cell Biol 34(7), 713-7; Amano, T., Tanabe, K., Eto, T., Narumiya, S., and Mizuno, K. (2001) Biochem J 354(Pt 1), 149-59; Edwards, D.C., Sanders, L. C., Bokoch, G. M., and Gill, G. N. (1999) Nat Cell Biol 1(5), 253-9; Maekawa, M., Ishizaki, T., Boku, S., Watanabe, N., Fujita, A., Iwamatsu, A., Obinata, T., Ohashi, K., Mizuno, K., and Narumiya, S. (1999) Science 285(5429), 895-8; Ohashi, K., Nagata, K., Maekawa, M., Ishizaki, T., Narumiya, S., and Mizuno, K. (2000) J Biol Chem 275(5), 3577-82; Sumi, T., Matsumoto, K., and Nakamura, T. (2001) J Biol Chem 276(1), 670-6; Bach, I. (2000) Mech Dev 91(1-2), 5-17; and Arber, S., Barbayannis, F. A., Hanser, H., Schneider, C., Stanyon, C. A., Bernard, O., and Caroni, P. (1998) Nature 393(6687), 805-9.

The disclosure of all references cited herein are incorporated by reference. The invention has been described in detail with reference to preferred embodiments thereof. However, it will be appreciated that those skilled in the art, upon consideration of this disclosure, may make modifications and improvements within the spirit and scope of the invention.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method of detecting an agent that modulates function of a membrane androgen receptor (mAR), the method comprising the steps of:
a. contacting the mAR with a detectably-labeled androgen compound under conditions that permit binding of the mAR and the compound to form a first complex,
b. contacting the first complex with the agent under conditions that permit binding between the agent and the mAR to form a second complex; and
c. detecting release of the detectably-labeled androgen compound from the first complex as being indicative of the presence of the agent.

2. The method of claim 1, wherein an increase in the release of the detectably labeled androgen compound, relative to a suitable control, is taken to be further indicative of the agent.

3. A method of detecting an agent that modulates function of a membrane androgen receptor (mAR), the method comprising the steps of:
a. contacting the mAR with a detectably-labeled androgen compound and the agent under conditions that permit binding of the mAR and the compound to form a first complex; and
b. detecting a decrease in the level of detectably-labeled androgen compound bound to the first complex as being indicative of the presence of the agent.

4. The method of claim 3, wherein the decrease in the level of bound androgen compound, relative to a suitable control, is taken to be further indicative of the agent that modulates mAR.

5. The method of claim 1, wherein the method further comprises the steps of:
i. contacting an intracellular androgen receptor (iAR) with a detectably-labeled androgen compound (the same or different) under conditions that permit binding of the iAR and the compound to form a third complex; and
ii. contacting the third complex with the agent under conditions that permit binding between the compound and the third complex.

6. The method of claim 1, wherein the method further comprises the step of contacting an intracellular androgen receptor (iAR) with a detectably-labeled androgen compound (the same or different) and the agent under conditions that permit binding of the iAR and the compound to form a third complex.

7. The method of claim 5, wherein the method is conducted essentially in parallel with the method of claim 1.

8. The method of claim 5, wherein the method further comprises the step of detecting negligible or no release of the detectably-labeled androgen compound from the third complex in the presence of the agent, relative to a suitable control, as being indicative of presence of the agent.

9. The method of claim 1, wherein one or more of the contacting steps is performed with a cell, cell line, or tissue sample that is known to or is suspected of expressing the mAR.

10. The method of claim 1, wherein the cell line that expresses the mAR is DU-145 or LNCaP.

11. The method of claim 1, wherein the tissue sample that expresses the mAR is prostate gland tissue.

12. The method of claim 11, wherein the prostate gland tissue is known or suspected to be cancerous.

13. The method of claim 5, wherein one or more of the contacting steps is performed with a cell, cell line or tissue sample expressing the iAR.

14. The method of claim 13, wherein the iAR is transiently expressed by the cell, cell line or a tissue sample.

15. The method of claim 14, wherein the cell or cell line further comprises a reporter gene construct comprising an androgen responsive element (ARE).

16. The method of claim 15, the method further comprising the step of detecting negligible or no signal from the reporter gene construct, relative to a suitable control, as being indicative of presence of the agent.

17. The method of claim 5, wherein iAR is present as a synthetic liposome composition.

18. The method of claim 1, wherein the method is performed using a subcellular fraction from cells, a cell line, or a tissue sample that are known or suspected to express the mAR.

19. The method of claim 1, wherein the method is performed using a subcellular fraction of cells that express the iAR.

20. The method of claim 1, wherein the androgen compound is detectably labeled with at least one of a radioisotope, a fluorophore, a quencher of fluorescence, an enzyme, an affinity tag, or a chemiluminescent, phosphorescent or chromophoric moiety.

21. The method of claim 20, wherein the androgen compound is detectably labeled with tritium.

22. The method of claim 1, wherein the detectably labeled androgen compound is tritiated dihydrotestosterone (³H-DHT) or tritiated testosterone (³H-T).

23. The method of claim 1, wherein the androgen compound comprises an androgen and a linker covalently bound to the androgen.

24. The method of claim 23, wherein the androgen is testosterone or dihydrotestosterone.

25. The method of claim 1, wherein the linker is bound to the 3′-carbon of the steroid ring, the linker being an O-carboxymethyl oxime linker.

26. The method of claim 1, wherein the detecting step is performed using at least one of: label displacement, surface plasmon resonance, fluorescence resonance energy transfer, fluorescence quenching, fluorescence polarization, luminescence, chemiluminescence, fluorescence, absorption, and scintillation counting.

27. The method of claim 1, wherein said agent is a peptide, peptoid, a polypeptide, an antibody or antigen-binding fragment thereof, a lipid, a carbohydrate, a nucleic acid, a small molecule; or a combination thereof.

28. The method of claim 27, wherein the agent is an androgen or a derivative thereof covalently bound to a linker and an amino acid sequence.

29. The method of claim 28 wherein the agent has the following structure covalently linked in sequence: testosterone or a derivative thereof/linker/serum albumin or a derivative thereof.

30. The method of claim 1, wherein the linker is a carboxy-methyl ether group.

31. The method of claim 30, wherein the agent is testosterone3-(O-carboxymethyl)oxime-serum albumin.

32. The method of claim 31, wherein the agent is detectably labelled.

33. The method of claim 1, wherein the method further comprises the step of detecting rapid modulation of a signaling molecule downstream of the mAR, the method further comprising the step of detecting a change in the level of the signaling molecule, relative to a suitable control.

34. The method of claim 33, wherein the step of detecting modulation of the signaling molecule comprises measuring activity of at least one of guanine nucleotide binding or exchange, kinase activity, guanosine triphosphatase activity, phosphatidylinosotol breakdown, diacylglycerol, inositol triphosphate, actin reorganization, reporter gene expression, and intracellular calcium level.

35. The method of claim 34, wherein the kinase is focal adhesion kinase (FAK) or phosphatidylinositol-3 (PI-3) kinase.

36. The method of claim 34, wherein the method further comprises detecting association of FAK with phosphatidylinositol-3 (PI-3) kinase.

37. The method of claim 34, wherein the guanosine triphosphatase is Cdc42/Rac1.

38. The method of claim 34, wherein the guanosine triphosphatase is RHO.

39. The method of claim 34, wherein the kinase is ROCK.

40. The method of claim 34, wherein the kinase is LIMK2.

41. The method of claim 34, wherein the intracellular calcium level is measured by using at least one of a calcium probe, an antibody to a calcium probe, a calcium ionophore and a calcium chelator.

42. The method of claim 1, wherein the method further comprises the step of rapidly detecting prostate specific antigen (PSA) expressed by the cells, cell line, or tissue samples known or suspected of expressing the mAR, iAR or both.

43. The method of claim 42, wherein the PSA is detected immunologically.

44. The method of claim 1, wherein the method further comprises the step of detecting cell death (apoptosis) in the cells, cell lines, or tissue samples that express the mAR.

45. The method of claim 44, wherein the apoptosis is detected by assaying an annexin or by using a detectable small molecule.

46. The method of claim 1, wherein the method further comprises the step of detecting any activation of the iAR in in vivo.

47. The method of claim 46, wherein the method further comprises administering the agent to a mammal subjected to an orchiectomy.

48. The method of claim 47, wherein the method further comprises selecting an agent with at least one of the following properties, relative to a suitable control, in the mammal:
a. negligible or no increase in prostate gland weight,
b. negligible or no increase in body weight,
c. negligible or no decrease in body fat; and
d. negligible or no increase in bone density.

49. The method of claim 48, wherein the method further comprises selecting an agent that has at least one of the foregoing properties as being further indicative of presence of the agent.
